(19) United States
(12) Patent Application Publication
(10) Pub. No.: US 2026/0144470 A1
(43) Pub. Date: May 28, 2026

(54) ESTIMATION DEVICE, AND ESTIMATION SYSTEM

(71) Applicant: TOYOTA JIDOSHA KABUSHIKI KAISHA, Toyota-shi (JP)

(72) Inventor: Tomohiro TSUBOTA, Nagoya-shi (JP)

(73) Assignee: TOYOTA JIDOSHA KABUSHIKI KAISHA, Toyota-shi (JP)

(21) Appl. No.: 19/350,551

(22) Filed: Oct. 6, 2025

(30) Foreign Application Priority Data
Nov. 22, 2024 (JP) 2024-203596

(51) Int. Cl. A61B 5/22; A61B 5/00; A61B 5/11; G01B 7/16

(52) U.S. Cl. CPC A61B 5/225; A61B 5/1114; A61B 5/6806; A61B 5/7267; A61B 5/7278; G01B 7/18; A61B 2503/20

(57) ABSTRACT

The estimation device comprises: an acquisition unit acquiring first a detection result obtained by a first sensor and a second detection result obtained by a second sensor, the first sensor being provided away from a palm side portion of a hand of a worker and detecting a first physical quantity related to bending of a finger of the worker, the second sensor being mounted on a forearm portion of the worker and detecting a second physical quantity related to a movement of muscle in the forearm portion; and an estimation unit estimating a finger force generated in the finger using the first detection result and the second detection result.

### CROSS REFERENCE TO RELATED APPLICATIONS

This application claims priority from Japanese Patent Application No. 2024-203596, filed Nov. 22, 2024, the disclosure of which is incorporated herein by reference in its entirety.

### BACKGROUND

### Field

This disclosure relates to an estimation device, and an estimation system.

### Related Art

JP 2023-167572 A discloses a wearable sensor having a sensor for measuring an operation related to a finger such as a movement of the finger.

In the technique of JP 2023-167572 A, at least a part of the sensor included in the wearable sensor is worn on the palm portion of the worker's hand. Due to the sensor attached to the palm portion, there was a possibility that the work by the worker was inhibited.

### SUMMARY

The present disclosure may be implemented in the form of the following aspects.

According to one aspect of the present disclosure, an estimation device is provided. The estimation device includes: an acquisition unit acquiring first a detection result obtained by a first sensor and a second detection result obtained by a second sensor, the first sensor being provided away from a palm side portion of a hand of a worker and detecting a first physical quantity related to bending of a finger of the worker, the second sensor being mounted on a forearm portion of the worker and detecting a second physical quantity related to a movement of muscle in the forearm portion; and an estimation unit estimating a finger force generated in the finger using the first detection result and the second detection result.

### BRIEF DESCRIPTION OF THE DRAWINGS

FIG. 1 is an explanatory diagram showing a schematic configuration of an estimation system;

FIG. 2 is a conceptual diagram illustrating the flow of a finger force's estimation;

FIG. 3 is a flow chart showing the process steps including an estimation process.

### DETAILED DESCRIPTION

### A. First Embodiment

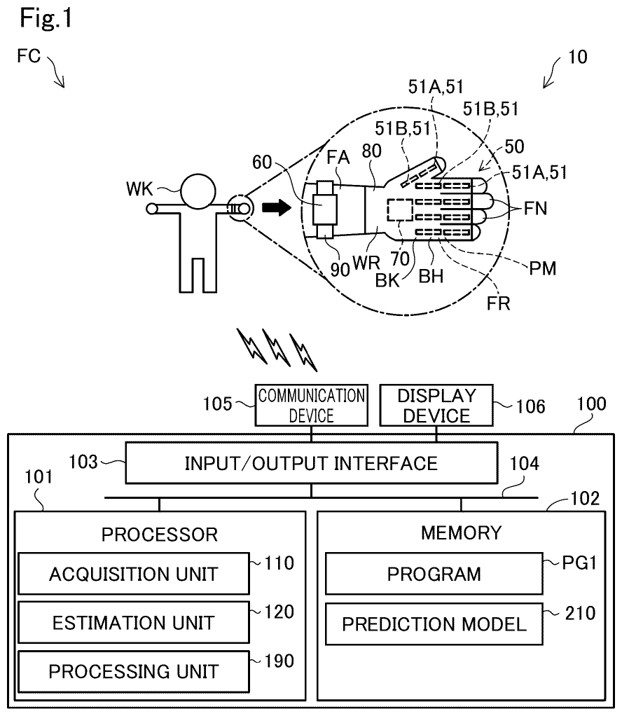

FIG. 1 is an explanatory diagram showing a schematic configuration of an estimation system 10 in the first embodiment. The estimation system 10 is used to estimate a finger force of a worker WK performing the Work. The finger force will be described later in more detail.

The estimation system 10 is used in a workshop where a worker WK performs the work. The workshop in the present embodiment is a factory FC for manufacturing a vehicle. The work in the present embodiment is a variety of work for manufacturing a vehicle, for example, work related to assembly of the vehicle, and work related to assembly of a part to the vehicle, work related to inspection of the vehicle. Such work may include, for example, removing, fitting, aligning, temporary placing, tightening, removing, binding, sticking, temporary placing, or placing. Such operations can also involve grasping the part by a hand of the worker WK or clamping the part by the finger FN of worker WK. Such work can also involve use of various tools by the worker WK. Such tools may be used, for example, gripped by the hand of the worker WK or clamped by the finger FN.

The above-described finger force represents a force generated in the finger FN of the worker WK in association with work performed by the worker WK. More specifically, the finger force is generated in association with gripping or pinching of a tool or a part by the worker WK. In the present embodiment, the estimation system 10 estimates magnitude of the finger force by executing an estimation process described below. The finger force may be estimated, for example, for each hand of the worker WK or for each finger FN of the worker WK. In the present embodiment, the finger force is estimated for each hand of the worker WK.

The estimation system 10 comprises a first sensor 50, a second sensor 60 and an estimation device 100. Furthermore, in the present embodiment, the estimation system 10 comprises a third sensor 70.

In the present embodiment, the first sensor 50, the second sensor 60, and the third sensor 70 are each configured as a wearable sensor. More specifically, the first sensor 50 and the third sensor 70 are mounted on a glove 80 wearable on a hand of the worker WK and are integrated into the glove 80. The second sensor 60 is mounted on a forearm portion FA of the worker WK via a band 90. The band 90 is configured to be worn on the forearm portion FA. In the present embodiment, the glove 80 has a shape not covering fingertip so as to expose the fingertip. However, in other embodiments, the glove 80 may have a shape covering the fingertip. The glove 80 may also be mounted, for example, overlaid on any other working glove worn on the hand of the worker WK.

The first sensor 50 is provided away from a palm side portion PM of the worker WK. The palm side portion PM includes not only a palm but also a palm side portion of the finger FN among the hand of worker WK. In the present embodiment, the first sensor 50 is provided on a body part of the worker WK that is different from the palm side portion PM. More specifically, the first sensor 50 is disposed on a back portion BK of the glove 80. The back portion BK of the glove 80 is a portion corresponding to a back side portion BH of the hand of the worker WK and is located on an opposite side to a front portion FR of the glove 80. The back side portion BH includes not only a dorsal portion of the hand, that is, a side opposite to a palm, but also back side portion of the finger of the hand of the worker WK. The front portion FR is a portion of the glove 80 that corresponds to the palm side portion PM of the worker WK. with this configuration, when the glove is worn on the hand of the worker WK, the first sensor 50 is attached to the back side portion BH of the worker WK.

The first sensor 50 detects a first physical quantity. The first physical quantity is a physical quantity related to bending of the finger FN of the worker WK. The detection result by the first sensor 50 is also referred to as the first detection result. The first detection result is associated with information representing the timing at which the first detection result is detected. Qqq first sensor 50 transmits the detected first detection result to the estimation device 100.

In the present embodiment, the first sensor 50 mechanically detects the first physical quantity. More specifically, in the present embodiment, the first sensor 50 is configured as a sensor group including a plurality of the bending sensors 51. In the present embodiment, for one finger FN, two bending sensors 51A,51B are arranged. Each bending sensors 51 is located along the skeleton of each finger FN. The bending sensor 51A is located between the bending sensor 51B and the fingertip in the back side portion BH. The bending sensor 51B is located between the bending sensor 51A and a wrist WR in the back side portion BH. In the present embodiment, the bending sensors 51 is configured as a resistance-type bending sensor and is configured such that an electrical resistance of the bending sensor 51 changes according to a bending degree of the bending sensor 51. With this configuration, the first sensor 50 mechanically detects, as the first physical quantity, a bending degree of each finger FN. In other embodiments, the bending sensor 51 may be configured as, for example, a capacitive bend sensor.

The second sensor 60 is mounted on the forearm portion FA of the worker WK. The second sensor 60 detects a second physical quantity. The second physical quantity is a physical quantity related to a movement of muscle of the forearm portion FA. A detection result obtained by the second sensor 60 is also referred to as a second detection result. The second detection result is associated with information representing timing at which the second detection result is detected. The second sensor 60 transmits the detected second detection result to the estimation device 100.

In the present embodiment, the second sensor 60 mechanically detects the second physical quantity. More specifically, in the present embodiment, the second sensor 60 is configured as a surface pressure sensor for detecting the movement of muscle of the forearm portion FA. The second sensor 60 as a surface pressure sensor, for example, may be configured as a resistance-type surface pressure sensor or may be configured as a capacitive surface pressure sensor. The second sensor 60 is sheet-shaped and has flexibility sufficient to be deformable along a shape of the forearm portion. The second sensor 60 detects, as a surface pressure distribution, a degree of muscular activity of each portion of the forearm portion FA associated with work by being attached so as to be in close contact with at least a part of the forearm portion FA. More specifically, muscles of the forearm portion FA contract or relax in association with work performed by the worker WK, and a degree of bulging or muscle stiffness of the muscles of the forearm portion FA changes, thereby changing a degree to which the second sensor 60 is pressed by the muscles of the forearm portion FA. The second sensor 60 detects changes in a degree of pressing by muscles of the forearm portion FA. With this configuration, the second sensor 60 mechanically detects, as the second physical quantity, a surface pressure distribution representing a degree of a muscular movement of the forearm portion FA. A technique for mechanically detecting or analyzing muscular activities such as contraction or relaxation of muscles and changes in a degree of bulging or muscle stiffness of the muscles associated therewith is also referred to as force myography (FMG). That is, in the present embodiment, the second sensor 60 is configured as a sensor capable of realizing FMG.

The second physical quantity detected as described above reflects the finger force as well as bending of the finger FN and wrist WR. That is, for example, even if the finger force is the same as each other, different second physical quantities can normally be detected when bending degrees of the fingers FN or a wrist WR are different.

The third sensor 70 is provided away from the palm side portion PM. In the present embodiment, the third sensor 70 is provided at a position of a body part of the worker WK that is different from the palm side portion PM. More specifically, the third sensor 70 is disposed on the back portion BK of the glove 80. With this configuration, when the glove 80 is worn on the hand of the worker WK, the third sensor 70 is attached to the back side portion BH. In the present embodiment, the third sensor 70 is disposed in the vicinity of the wrist WR of the worker WK in the back side portion BH. More specifically, the third sensor 70 is disposed between the first sensor 50 and the wrist WR.

The third sensor 70 detects a third physical quantity. The third physical quantity is a physical quantity related to bending of the wrist WR of the worker WK. A detection result by the third sensor 70 is also referred to as a third detection result. The third detection result is associated with information representing timing at which the third detection result is detected. The third sensor 70 transmits the detected third detection result to the estimation device 100. In other embodiments, for example, a transmission unit for transmitting the first detection result and the third detection result in an aggregated manner to the estimation device 100 may be mounted on the glove 80, and the first detection result and the third detection result may be transmitted to the estimation device 100 via the transmission unit.

In the present embodiment, the third sensor 70 dynamically detects the third physical quantity. More specifically, the third sensor 70 is configured as an inertial measuring device (IMU) including a three-axis acceleration sensor, a three-axis gyro sensor, and a three-axis geomagnetic sensor. As the third physical quantity, the third sensor 70 dynamically detects the accelerations and angular velocities on the wrist WR of the worker WK. The position and angular velocity of the wrist WR of the worker WK can be obtained by using the integration of the detected accelerations and angular velocities. Furthermore, it is possible to acquire a bending degree of the wrist WR based on the position and an angle of the wrist WR.

In the present embodiment, the first sensor 50 and the third sensor 70 are mounted on the back side portion BH, and the second sensor 60 is mounted on the forearm portion FA, so that none of the sensors, such as the first sensor 50, the second sensor 60, or the third sensor 70, are mounted on the palm side portion PM.

The estimation device 100 is configured as a computer with a processor 101, a memory 102 including ROM and RAM, an input/output interface 103, and an internal bus 104. The processor 101, the memory 102, and the input/output interface 103 are connected to be able to communicate in both directions via the internal bus 104. The input/output interface 103 is connected to the communication device 105 and the display device 106. The communication device 105 may communicate directly or indirectly with the first sensor 50, the second sensor 60, and the third sensor 70 via wired or wireless communication. The display device 106 is configured as, for example, a liquid crystal display or the like, and displays various information such as information related to an estimated result by the estimation system 10. The memory 102 stores various information such as a program PG1 and a prediction model 210. The processor 101 implements various functions, including functions as an acquisition unit 110, an estimation unit 120, and a processing unit 190, by executing a program PG1.

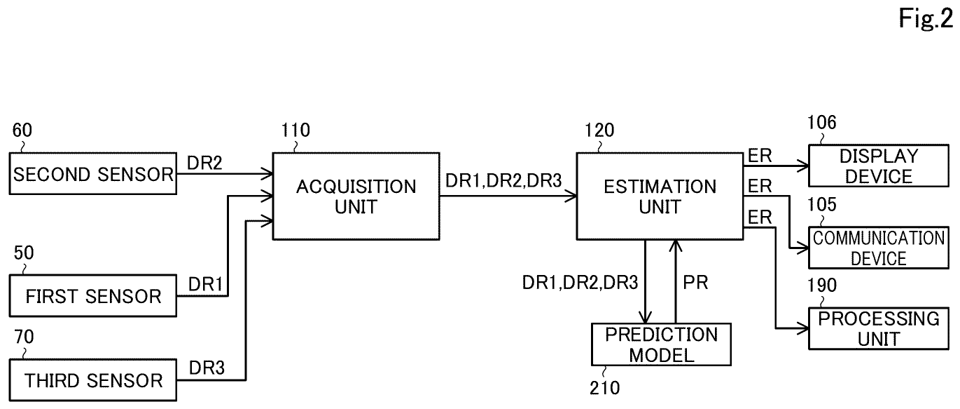

FIG. 2 is a conceptual diagram illustrating a flow of estimation of the finger force in the present embodiment. As shown in FIG. 2, the acquisition unit 110 acquires the first detection result DR1 and the second detection result DR2. In the present embodiment, the acquisition unit 110 further acquires the third detection result DR3.

The estimation unit 120 executes an estimation process. The estimation process is a process of estimating the finger force of the worker WK using the first detection result DR1 and the second detection result DR2 acquired by the acquisition unit 110. In the estimation process according to the present embodiment, the estimation unit 120 estimates the finger force of the worker WK by further using the third detection result DR3 acquired by the acquisition unit 110. The estimation unit 120 records the finger force estimated by the estimation process in the memory 102 as an estimation result ER. The estimation unit 120 outputs the estimation result ER. More specifically, the estimation unit 120 causes an estimation result ER to be displayed on a display device 106, and causes a processing unit 190 to execute subsequent processing described below by outputting the estimation result ER.

In the present embodiment, the estimation unit 120 estimates the finger force using the prediction model 210. The prediction model 210 is a machine-learning model trained to provide a prediction result PR of the finger force based on the first detection result DR1, the second detection result DR2, and the third detection result DR3. in the present embodiment, a prediction model 210 is trained to output a prediction result PR of the finger force by using information including a first detection result DR1, a second detection result DR2, and a third detection result DR3 as input.

In the present embodiment, the prediction model 210 has been trained by supervised learning using a training dataset. The training dataset includes a plurality of training data and a plurality of labels. In the training dataset, each training data is associated with each label. The training data correspond to explanatory variables, and the labels correspond to objective variables. In the present embodiment, as the training data, data including information representing the first detection result DR1, the second detection result DR2, and the third detection result DR3 is used. As the label, magnitude of the finger force is used. The training dataset is prepared, for example, by measuring a grip force corresponding to the finger force using a conventional grip force meter while measuring the first physical quantity, the second physical quantity, and the third physical quantity while the sensors are worn on the worker WK. As the prediction model 210, for example, various machine learning models such as a random forest, a support vector machine (SVM), and a neural network can be used. In other embodiments, the learning method of the prediction model 210 is not limited to supervised learning. For example, the prediction model 210 may have been trained by unsupervised learning or reinforcement learning.

The processing unit 190 performs a subsequent process using the estimation result ER in the estimation system 10. The subsequent process is a process for exploiting the estimation result ER. The subsequent process includes, for example, an analysis process for analyzing the estimation result ER. In the analysis process, the processing unit 190 analyzes, in real time or retrospectively, appropriateness of a state of the worker WK and appropriateness of a manner of work by the worker WK by comparing the finger force as the estimation result ER with a reference finger force predetermined according to the work. Such analysis process may be used, for example, for quality assurance of products produced in the workshop or for safety evaluation of work in the workshop. The processing unit 190 may cause a processing result of the subsequent process to be displayed on a display device 106, for example. The content of the subsequent process is not limited to the above.

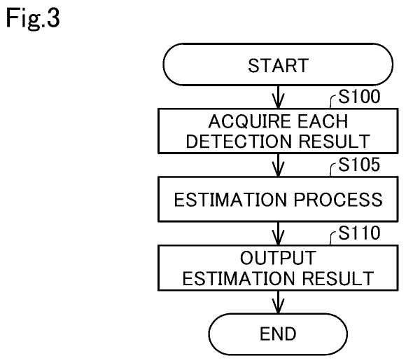

FIG. 3 is a flow chart showing a process sequence including the estimation process in the present embodiment. The process steps shown in FIG. 3 are performed by the processor 101 of the estimation device 100 at predetermined time-intervals, for example.

In step S100 of FIG. 3, the acquisition unit 110 acquires each detection result by each sensor. More specifically, in the step S100, the acquisition unit 110 acquires the first detection result DR1, the second detection result DR2, and the third detection result DR3. In step S105, the estimation unit 120 executes the estimation process. More specifically, in step S105 in the present embodiment, the estimation unit 120 estimates the finger force by inputting each detection result acquired in step S100 into the prediction model 210 and cause the prediction model 210 to output the prediction result PR of the finger force. In step S105, the estimation unit 120 records the estimated finger force as the estimation result ER in the memory 102. In the step S110, the estimation unit 120 outputs the estimation result ER.

According to the estimation device 100 in the present embodiment described above, the finger force of the worker WK is estimated by using the first detection result DR1 obtained by the first sensor 50 and the second detection result DR2 obtained by the second sensor 60. The first sensor 50 is provided away from the palm side portion PM and detects the first physical quantity related to bending of the finger FN. The second sensor 60 is mounted on the forearm portion FA and detects the second physical quantity related to the movement of the muscle of the forearm portion FA. In this way, since it is not required to attach a sensor to the palm side portion PM, it is possible to suppress interference with gripping of a tool or a part by the hand of the worker WK or pinching of the tool or the component by the fingers FN due to a sensor attached to the palm side portion PM. As a result, it is possible to suppress interference with work performed by the worker WK due to the sensor. Unlike the present embodiment, for example, it is difficult to estimate the finger force using only the second detection result reflecting the finger force and the degree of bending of the finger FN or to estimate the finger force using only the first detection result DR1 simply reflecting the degree of bending of the finger FN. In contrast, in the present embodiment, the finger force can be appropriately estimated by using the first detection result DR1 and the second detection result DR2.

In the present embodiment, for example, as compared with the case where a pressure sensor or a load sensor for directly detecting the finger force is provided on the palm side portion PM, direct contact between the sensor and the part or the tool caused by the work can be suppressed, and damages of the sensor can be suppressed. Furthermore, for example, as compared with the case of providing a protective structure for the purpose of suppressing the damages of such sensors on the front portion FR of the glove 80, it is possible to suppress the thickness of the front portion FR is increased, and it is possible to suppress the work is inhibited due to the thickness of the front portion FR.

In the present embodiment, the finger force is estimated by using the third detection result DR3 by the third sensor 70. The third sensor 70 is provided away from the palm side portion PM and detects the third physical quantity related to bending of the wrist WR of the worker WK. In this way, by using the third detection result DR3 in addition to the first detection result DR1 and the second detection result DR2, it is possible to estimate finger force more effectively while suppressing interference with work performed by the worker WK due to the sensor. More specifically, for example, even when the estimation process is executed in a situation where a bending degree of the wrist WR of the worker WK can vary depending on an estimation timing at which the finger force is estimated, it is possible to estimate the finger force with high accuracy.

In the present embodiment, the first sensor 50 and the third sensor 70 are mounted to the back side portion BH. In this way, the first sensor 50 and the third sensor 70 are integrated into the hand of the worker WK, and it is possible to suppress the work of the worker WK from being inhibited due to the sensor. Furthermore, as in the present embodiment, the first sensor 50 and the third sensor 70 can be compactly integrated into a hand-worn attachment such as the glove 80.

In the present embodiment, the first sensor 50 and the third sensor 70, respectively, dynamically detect the first physical quantity and the third physical quantity. Thus, for example, as compared with the case where the first sensor 50 and the third sensor 70 is configured to detect each physical quantity optically, it is possible to suppress the detection of each physical quantity is inhibited by disturbances such as foreign matter, and it is possible to detect each physical quantity with higher robustness. Consequently, the finger force can be estimated with higher robustness.

In the present embodiment, the estimation unit 120 estimates the finger force based on the first detection result DR1, the second detection result DR2, and the third detection result DR3 by using the prediction model 210 that has already been trained to output the prediction result PR of the finger force. Therefore, the finger force can be estimated by integrating the first detection result DR1, the second detection result DR2, and the third detection result DR3 using a simple method.

### B. Other Embodiments

(B1) In the above embodiment, although the estimation unit 120 uses the third detection result DR3 in the estimation process, the third detection result DR3 may not be used. In other words, the estimation unit 120 may estimate the finger force using at least the first detection result DR1 and the second detection result DR2 in the estimation process. In this case, the estimation unit 120 may estimate finger force by using a machine learning model trained to predict the finger force based on the first detection result DR1 and the second detection result DR2, for example. also in such a configuration, for example, when the estimation process is executed in a situation where a bending degree of a wrist WR of the worker WK does not change depending on an estimation timing or in a situation where a change in the bending degree depending on the estimation timing is relatively small, it is possible to estimate the finger force with high accuracy. In addition, in a configuration where the third detection result DR3 is not used in the estimation process as described above, the acquisition unit 110 does not necessarily acquire the third detection result DR3. In this configuration, the estimation system 10 does not necessarily include the third sensor 70.
(B2) In the above embodiment, the first sensor 50 is configured as the sensor group including a plurality of bending sensors 51, and the third sensor 70 is configured by IMU, but is not limited thereto. For example, the first sensor 50 may be configured by an IMU. The third sensor 70 may be configured by, for example, one or more bending sensors. In the above embodiment, the first sensor 50 and the third sensor 70, respectively, detect the first physical quantity and the third physical quantity mechanically, but not limited thereto. For example, the first sensor 50 and the third sensor 70 may be configured as optical sensors for optically detecting the respective physical quantities. The optical sensor includes, for example, a camera and a light detection and ranging (Lidar) devices. In this case, the functions as the first sensor 50 and the third sensor 70 may be realized by, for example, one optical sensor.
(B3) In the above embodiment, the first sensor 50 and the third sensor 70 are mounted on the back side portion BH. In contrast, the first sensor 50 and/or the third sensor 70 may not be attached to the back side portion BH if the sensor(s) are provided away from the palm side portion PM. For example, the first sensor 50 and/or the third sensor 70 may be mounted on a side portion of the hand of the worker WK. The side portion of the hand includes a side portion of the hand and a side portion of finger FN. The first sensor 50 and/or the third sensor 70 configured as an optical sensor may be attached to a body part other than the hand or an arm of the worker WK, or may be provided away from the worker WK.
(B4) In the above embodiment, the machine learning model is used in the estimation process, but the machine learning model may not be used. For example, in the estimation process, the finger force may be estimated by using a pre-prepared rule-based system. Such a rule-based system may be configured, for example, to calculate an index value corresponding to a component derived from the finger force by subtracting a component derived from bending of the finger FN from an operation value (for example, a value representing the muscle stiffness) representing the degree of movement of the muscle of the forearm portion FA, and to output a predicted value of the finger force based on the calculated index value. In this case, when calculating the index value, from the operation value, further, components derived from the bending of the wrist WR may be subtracted. In this case, the operating value is calculated based on the second detection result DR2. The components derived from the bending of the finger FN are calculated based on the first detection result DR1. The components derived from the bending of the wrist WR are calculated based on the third detection result DR3.
(B5) In the above-described embodiment, the prediction model 210 is a machine learning model that has been trained to output the prediction result PR by using a first detection result DR1, a second detection result DR2, and a third detection result DR3 as input. In contrast, the prediction model 210 only needs to be configured to output the prediction result PR based on the first detection result DR1, the second detection result DR2, and the third detection result DR3, and does not necessarily use the first detection result DR1, the second detection result DR2, and the third detection result DR3 as input. For example, the prediction model 210 may be configured to use a predicted value based on the first detection result DR1 instead of the first detection result DR1 as input. The prediction model 210 may also be configured to use a predicted value based on the second detection result DR2 instead of the second detection result DR2 as input. The prediction model 210 may also be configured to use a predicted value based on the third detection result DR3 instead of the third detection result DR3 as input. The predicted value input to the prediction model 210 may be output by using one or more machine learning models different from the prediction model 210, or may be output by using a rule-based system. Such machine learning models may be trained to output a predicted value by using one or two of the first detection result DR1, the second detection result DR2, and the third detection result DR3 as input. The estimation unit 120 may be configured to change the prediction model 210 to be used according to one or two of the first detection result DR1, the second detection result DR2, and the third detection result DR3, for example. For example, the estimation unit 120 may switch between two prediction models 210 that output the prediction result PR by using the second detection result DR2 and the third detection result DR3 as input depending on whether a bending degree of the fingers FN as the first detection result DR1 is equal to or greater than a predetermined degree or is less than the predetermined degree. Even in such a configuration, the prediction model 210 can output the prediction result PR based on the first detection result DR1, the second detection result DR2, and the third detection result DR3.
(B6) In the above embodiment, the surface pressure sensor is used as the second sensor 60, but is not limited thereto. For example, as the second sensor 60, various sensors for implementing FMG may be used. As the second sensor 60, for example, various piezoelectric sensors or various capacitive sensors may be used. The shapes and materials of such the second sensor 60 may be optional. For example, a functional rubber material may be used for the second sensor 60, or a functional fiber material capable of realizing a smart textile (E-Textile) technique may be utilized. Similarly to the second sensor 60, a functional rubber material may be used for the first sensor 50 and the third sensor 70, and E-textile technology may be utilized for the first sensor 50 and the third sensor 70. The second sensor 60 is not limited to the sensor that realizes FMG, and for example, an EMG sensor that can detect the movement of the muscle of the forearm portion FA using electromyography (EMG) may be used. The EMG sensor has an electrode for detecting electrical signals generated in muscles in association with muscular activity, and detects the second physical quantity by using the electrode.

The disclosure is not limited to any of the embodiment and its modifications described above but may be implemented by a diversity of configurations without departing from the scope of the disclosure. For example, the technical features of any of the above embodiments and their modifications may be replaced or combined appropriately, in order to solve part or all of the problems described above or in order to achieve part or all of the advantageous effects described above. Any of the technical features may be omitted appropriately unless the technical feature is described as essential in the description hereof. The present disclosure may be implemented by aspects described below.
(1) According to one aspect of the present disclosure, an estimation device is provided. The estimation device includes: an acquisition unit acquiring first a detection result obtained by a first sensor and a second detection result obtained by a second sensor, the first sensor being provided away from a palm side portion of a hand of a worker and detecting a first physical quantity related to bending of a finger of the worker, the second sensor being mounted on a forearm portion of the worker and detecting a second physical quantity related to a movement of muscle in the forearm portion; and an estimation unit estimating a finger force generated in the finger using the first detection result and the second detection result.

According to this aspect, since it is not necessary to attach the sensor to the palm side portion of the hand of the worker, the finger force can be appropriately estimated using the first detection result and the second detection result while suppressing inhibition of work performed by the worker due to the sensor.
(2) In the above-described aspect, the acquisition unit may further acquire a third detection result obtained by a third sensor, the third sensor being provided away from the palm side portion and detecting a third physical quantity related to bending of a wrist of the worker. The estimation unit may estimate the finger force by further using the third detected result.

According to this aspect, the finger force can be more effectively estimated by using the third detection result in addition to the first detection result and the second detection result.
(3) In the above-described aspect, the first sensor and the third sensor may be mounted on a back side portion of the hand of the worker.

According to this aspect, it is possible to suppress interference with work performed by the worker due to the sensors while integrating the first sensor and the third sensor into the hand of the worker.
(4) In the above embodiment, the estimation unit may estimate the finger force by using a machine learning model trained to output a prediction result of the finger force based on the first detection result, the second detection result, and the third detection result.

According to this aspect, it is possible to estimate the finger force by integrally using the first detection result, the second detection result, and the third detection result with a simple method.

The present disclosure can be implemented in various aspects other than the estimation device described above. For example, the present disclosure may be embodied in aspects of an estimation system, an estimation method, a program for realizing the estimation method, non-transitory storage mediums storing the program, and a program product. The program product may be provided, for example, as a non-transitory recording medium on which the program is recorded, or as a program product distributed via a network.

What is claimed is:

1. An estimation device, comprising:
an acquisition unit acquiring first a detection result obtained by a first sensor and a second detection result obtained by a second sensor, the first sensor being provided away from a palm side portion of a hand of a worker and detecting a first physical quantity related to bending of a finger of the worker, the second sensor being mounted on a forearm portion of the worker and detecting a second physical quantity related to a movement of muscle in the forearm portion; and
an estimation unit estimating a finger force generated in the finger using the first detection result and the second detection result.

2. The estimation device according to claim 1, wherein
the acquisition unit further acquires a third detection result obtained by a third sensor, the third sensor being provided away from the palm side portion and detecting a third physical quantity related to bending of a wrist of the worker,
the estimation unit estimates the finger force by further using the third detected result.

3. The estimation device according to claim 2, wherein
the first sensor and the third sensor are mounted on a back side portion of the hand of the worker.

4. The estimation device according to claim 3, wherein
the estimation unit estimates the finger force by using a machine learning model trained to output a prediction result of the finger force based on the first detection result, the second detection result, and the third detection result.

5. An estimation system, comprising:
a first sensor being provided away from a palm side portion of a hand of a worker and detecting a first physical quantity related to bending of a finger of the worker;
a second sensor being mounted on a forearm portion of the worker and detecting a second physical quantity related to a movement of muscle in the forearm portion;
an acquisition unit acquiring a first detection result obtained by the first sensor and a second detection result obtained by the second sensor; and
an estimation unit estimating a finger force generated in the finger using the first detection result and the second detection result.
